(19) United States
(12) Patent Application Publication
(10) Pub. No.: US 2024/0277499 A1
(43) Pub. Date: Aug. 22, 2024

(54) MEDICAL DEVICE AND TREATMENT SYSTEM

(71) Applicant: Acandis GmbH, Karlsruhe (DE)

(72) Inventor: Alexander Lange, Karlsruhe (DE)

(21) Appl. No.: 18/443,462

(22) Filed: Feb. 16, 2024

(30) Foreign Application Priority Data
Feb. 20, 2023 (DE) 102023104170.3

(51) Int. Cl. A61F 2/915; A61F 2/00

(52) U.S. Cl. CPC A61F 2/915; A61F 2002/0081; A61F 2002/91541; A61F 2002/91583; A61F 2230/0017; A61F 2230/0028

(57) ABSTRACT

A medical device for insertion into an organ of the body, with a compressible and expandable mesh structure having struts which are connected together by strut connectors and delimit closed cells of the mesh structure, wherein respectively, two of the struts of at least one cell are disposed opposite each other and form a first strut pair and a second strut pair, wherein one of the two strut pairs is connected together by at least one connecting strut which extends into the cell and bridges it.

### CROSS-REFERENCE TO RELATED APPLICATION

The present patent application claims the benefit of and priority to German Patent Application No. 10-2023-104170.3, filed Feb. 20, 2023, the entire contents of which are incorporated herein for all purposes by reference.

### FIELD

The invention relates to a medical device for insertion into an organ of the body, with a compressible and expandable mesh structure comprising struts. The struts are connected together by strut connectors and delimit closed cells of the mesh structure. Respectively, two struts of at least one cell are disposed opposite each other and form a first strut pair and a second strut pair.

### BACKGROUND

A medical device of this type is known from EP 1 903 999 B1, for example, which describes a stent which has a closed cell design. In stents of this type, the problem arises that in tightly curved vessels, they do not always have sufficient bending flexibility to remain in good contact with the vessel wall (wall apposition). The measures for improving the bending flexibility taken in the prior art mentioned above are complex.

The objective of the invention is to improve the bending flexibility of the mesh structure of a medical device in a simple manner without relinquishing the closed celled design.

### SUMMARY

In accordance with the invention, this objective is achieved by a medical device as shown in the drawings and described herein.

A further objective of the invention is to provide a treatment system with a medical device of this type. This objective is achieved as shown in the drawings and described herein.

Specifically, the objective is achieved by a medical device for insertion into an organ of the body, with a compressible and expandable mesh structure comprising struts. The struts are connected together by strut connectors and delimit closed cells of the mesh structure.

Respectively, two struts of at least one cell are disposed opposite each other and form a first strut pair and a second strut pair. One of the two strut pairs is connected together by at least one connecting strut. The connecting strut extends into the cell and bridges it.

The invention has a variety of advantages.

The connecting strut meets the constructional prerequisites for optimizing the mesh structure with closed cells as regards the bending flexibility, without at the same time compromising or at least substantially compromising the supporting force or radial force of the mesh structure. The invention permits very good vessel apposition to be obtained in tortuous vessels with a high supporting force or radial force. Risks which may arise from poor wall apposition, such as migration or negative hemodynamic effects, are considerably mitigated. To this end, at least one connecting strut is provided which connects one of the two strut pairs together, extends into the cell and bridges it. By means of the additional connecting strut, an increased supporting force is obtained, so that other properties or features of the mesh structure can be modified in order to improve the bending flexibility, such as the number of cells on the circumference, for example, without increasing the risk of migration.

The closed cell design has the advantage of good resheathability for repositioning the device in the vessel.

The additional connecting strut is simple to produce, for example by laser cutting. The good re-positionability of the mesh structure is not compromised.

A further advantage of the invention lies in the fact that the basic shape of the mesh structure can be retained. The shape of the cells as a closed cell can be retained. The additional connecting strut supplements the cell design. The “bridging” of the cell by the connecting strut should be understood to mean that the design of the basic cell additionally includes the at least one connecting strut which extends over the entire basic cell between two struts which are disposed opposite each other. The connecting strut bridges or spans the entire cell and connects the two opposite struts of a strut pair.

In general, the opposing struts of a (first) strut pair are not connected together directly, but each to the opposite struts of a further (second) strut pair as well as to the connecting strut.

Together with the further strut pair, the strut pair forms the boundary of the cell. This does not exclude the possibility that the cell could be delimited by further struts. It is also possible for more than one connecting strut to protrude into the cell or into the respective cell and bridge it.

The opposing struts of the strut pairs may be straight and/or curved. Reference should be made to the Applicant's patents in respect of the basic design of the cells.

EP 2 667 831 B1 protects the underlying principle of rotation during expansion via paired opposing struts with different bending stiffnesses. DE 10 2013 104 550 B4 describes a cell design and the shape of the strut connectors, wherein the strut connectors are X-shaped. Because respective adjacent struts have unequal strut widths, twisting of the stent structure occurs during expansion. DE 10 2013 107 258 B4 additionally describes a complete stent construction with edge and transitional cells, cross sectional shapes with flaring as well as edge cells with markers.

The invention is applicable to these protected technologies and is disclosed and claimed in conjunction with the cell designs described in the Applicant's own patents. This does not exclude the fact that the invention is applicable to other cell designs which have not been described here.

The principle of the invention applies to at least one cell of the mesh structure. The principle may be broadened to a plurality of cells of the mesh structure, in particular to a plurality of cells disposed on the circumference of the mesh structure. The present features which are described and claimed in conjunction with at least one cell are also described and claimed in conjunction with a plurality of cells, in particular with a plurality of cells disposed on a segment of the circumference.

The device in accordance with the invention is, for example, suitable as a temporary or permanent vessel implant. It may, for example, be a stent or a thrombectomy device. Other applications are conceivable.

Advantageously, the mesh structure is substantially tubular.

The mesh structure may be self-expanding.

Preferred embodiments of the invention are defined in the dependent claims.

Preferably, the cell is substantially rhomboidal. This has the advantage that the connecting strut is used in conjunction with a well-established closed cell shape which can be optimized well with a view to the bending flexibility. “Rhomboidal” is not used in the strictly geometrical sense in which the struts have to be straight. It is sufficient for the basic shape of the cell to be rhomboidal. The respective strut pairs may each be straight and/or curved opposing struts.

In a preferred embodiment, a plurality of cells are disposed on at least one circumferential segment in which respectively one of the two strut pairs is connected together by at least one connecting strut which extends into the cell and bridges it. Transferring the principle of one cell to a plurality of cells of a circumferential segment has the effect that the supporting force is increased by the additional connecting struts of the respective cells on the entire circumference in the region of the circumferential segment. The number of cells on the circumferential segment is not restricted. It is possible for there to be a plurality of circumferential segments in the axial direction, which respectively have appropriate cells each with at least one connecting strut.

The circumferential segment with the cells with connecting struts can be described as the first circumferential segment.

In a preferred embodiment, the strut connectors of the first circumferential segment are offset on the circumference by an angle which is less than or equal to 180°. This has the advantage that the mesh structure—in the case of a tubular mesh structure—can be compressed to a particularly small diameter when crimped in a catheter. The strut connectors are in a particularly tight arrangement because of the offset disposition, whereupon small diameters of the mesh structure in the crimped state can be obtained.

As an example, three cells disposed on the circumferential segment, in particular the first circumferential segment, are configured in a manner such that the strut connectors-viewed in cross section of the mesh structure—are offset by 120°. The invention or the present exemplary embodiment is not limited to three cells per circumferential segment. The offset arrangement of the strut connectors with respect to the cross section of the mesh structure may also be obtained with another number of cells.

The “cross section” of the mesh structure should be understood to mean a section which runs orthogonally to the longitudinal axis of the mesh structure.

In a particularly preferred embodiment of the invention, the connecting strut bridges the cell diagonally. In this way, a particularly high supporting force is achieved.

The diagonal disposition of the connecting strut is disclosed and claimed in conjunction with at least one cell, in particular in conjunction with all of the cells of the first circumferential segment. A plurality of circumferential segments may be provided in the axial direction which are equipped with diagonal connecting struts corresponding to the first circumferential segment.

In this regard, the “diagonal disposition” means that, with respect to at least one axis of the cell, in particular both axes of the cell, the connecting strut is inclined to, or in general deviates from the position of the axis or the axes, i.e. the connecting strut intersects the axis or the axes. As an example, in the case of a rhomboidal cell, the longitudinal axis of the cells runs between the two tips of the rhomboidal cell which have the larger separation. The normal axis is shorter than the longitudinal axis. The position of the axes may change with respect to the longitudinal axis of the entire device as a function of the state of expansion or compression.

The diagonal disposition means that the connecting strut only connects one of the two strut pairs together. The other strut pair is free having regard to the connecting strut.

The diagonally disposed connecting strut may be straight or curved in configuration. The curved connecting strut is advantageously S-shaped in configuration. Other shapes for the connecting strut are possible.

Preferably, the connecting strut is connected to the strut pair by a respective flexible strut connector, in particular a Z-shaped strut connector. The flexible strut connector, in particular the Z-shaped strut connector, has a greater flexibility than X-shaped strut connectors. This has the advantage that the bending flexibility is further improved.

The flexible strut connector permits relative movement between the connecting strut and the strut pair when the mesh structure is moved, for example curved or expanded/compressed. As an example, the flexible strut connector may be Z-shaped.

In a further advantageous embodiment, the connecting struts of a first cell and the connecting struts of an adjacent second cell in the circumferential direction are offset with respect to each other. An example of how the offset disposition of the connecting struts of two adjacent cells may be obtained is the connection of the respective connecting strut with the associated strut pair via a Z-shaped strut connector.

Other possibilities for the attachment of the connecting struts which cause the connecting struts of two cells which are adjacent in the circumferential direction to be offset with respect to each other are conceivable.

The Z-shaped attachment of the connecting struts ensures that the diagonal connecting struts of two adjacent cells are not aligned, but that their connecting sites with the cell, in particular the rhomboidal cell, are offset with respect to each other in a manner such that in the loaded state, i.e. in the compressed state in the catheter, parts of these adjacent diagonally disposed connecting struts overlap.

In this regard, the offset between the junction sites or connection sites of the diagonally disposed connecting struts to the respective strut pairs, i.e. the length of the overlap, is between 0.1 and 0.9 times the strut length of the cell, in particular of the rhomboidal cell. The offset may be between 0.2 and 0.8 times, in particular between 0.3 and 0.7 times the strut length of the cell, in particular of the rhomboidal cell.

Preferably, the strut width of the connecting strut in the region of its ends is smaller than the strut width in a region which extends between the ends of the connecting strut. In this manner, wall adaptation is further improved, because the diagonal strut of the cell which lies on the inside of a bend in a vessel and is therefore compressed, then has a lower tendency to bend into the interior of the vessel due to the compression.

More preferably, the strut width of the struts of the first and/or second strut pair in a region close to the X-shaped strut connectors is smaller than the strut width in a region close to the Z-shaped strut connectors. The struts of the first and/or second strut pair here preferably taper in configuration in the direction of the X-shaped strut connectors. Good bending flexibility of the struts or of the mesh structure is obtained in this manner.

The ratio of the strut width of the struts in the region close to the X-shaped strut connectors to the strut width close to the Z-shaped strut connectors is preferably between 0.5 and 1.0, in particular approximately 0.75. The ratio of the strut width in the region of the ends of the connecting strut to the strut width in the region between the ends of the connecting strut is between 0.7 and 1.0, in particular approximately 0.95. The struts of the first and/or second strut pair in the region close to the X-shaped strut connectors are consequently more sharply tapered than the ends of the connecting struts.

In a specific embodiment, the circumferential segment has at least 2, in particular 3 cells. In this manner, the bending flexibility is improved compared with an embodiment with more cells on the circumference. Preferably, the mesh structure has a maximum of 5 cells per circumferential segment. The edge regions may have more than 5 cells per circumferential segment, for example 6 cells. Other designs are possible.

The number of cells on the first circumferential segment is advantageously smaller than the number of cells on at least one second circumferential segment which is disposed proximally and/or distally to the first circumferential segment in the axial direction of the mesh structure. In this embodiment, a plurality of circumferential segments are provided in the axial direction of the mesh structure, wherein the circumferential segments which are disposed further outwards relative to the first circumferential segment or a further outwardly disposed circumferential segment has more cells on the circumference than the first circumferential segment.

This embodiment has the advantage that the further inwardly disposed cells in the axial direction of the mesh structure, which may also be described as central cells or inner cells, can be properly optimized with a view to the bending flexibility, because the central cells each have connecting struts, so that in this region, the flexibility is particularly high, but the supportive effect is not especially reduced because the radial force is distributed evenly onto the surface of the vessel.

In contrast to the central cells, the outer cells or outward cells or edge cells which are further out relative to the first circumferential segment or to the central cells do not have any connecting struts. The outward cells may have other functions or properties, such as flaring or radiopacity by means of radiographic markers, for example.

Transposed onto a stent or a thrombectomy device, this embodiment has the advantage that in the central region of the mesh structure, where particularly tight curves occur, the cells of the mesh structure are reinforced by the additional connecting struts so that there, the bending flexibility can be optimized. In this embodiment in accordance with the invention, this is achieved by the fact that the number of rhomboidal cells on the first circumferential segment is smaller than the number of cells on at least one second circumferential segment which is disposed proximally and/or distally to the first circumferential segment in the axial direction of the mesh structure.

The associated poorer distribution of the supporting effect which is usually associated with a smaller number of cells is compensated for by reinforcing these cells by means of the additional connecting struts, so that in this region of the mesh structure, a very good bending flexibility is obtained at the same time as a well-distributed supporting force. The distribution of the supporting force over a larger number of struts compared to the circumferential segment with the same number of cells without connecting struts means that the supporting force is not concentrated onto a few lines of contact, or put another way, it is transferred to a plurality of lines of contact. The connecting struts also contribute to increasing the supporting force.

The ratio of the number of central cells per circumferential segment to the number of outer cells per circumferential segment may be 1:1, 1:2 or 1:3 or 1:4. Other ratios are possible with this embodiment, as long as fewer central cells are provided per circumferential segment than outer cells per circumferential segment.

The basic principle of the invention may also be embodied in an embodiment which has a constant number of cells per circumferential segment along the entire length of the mesh structure.

Here, for the same number of cells, the cell design of the outer cells and the cell design of the central cells may be different. Other differences are possible.

The central cells may also be described as middle cells or inner cells and the outer cells may also be described as outward cells. It is simply a matter of the relative disposition of the cells with respect to each other.

The basic principle of the invention may also be applied to a medical device which has a membrane, wherein the membrane at least partially covers the cells of the mesh structure. The membrane may be configured as an electrospun membrane. In this case, preferably, 9 cells are provided per circumferential segment, because in this manner, the inflow of blood between the vessel wall and the membrane can be substantially prevented or reduced.

The treatment system has a medical device in accordance with the invention or a medical device according to one of the embodiments defined above as well as a catheter in which a guide element is connected to an axial end of the mesh structure.

### BRIEF DESCRIPTION OF THE DRAWINGS

The invention will now be described in further detail with the aid of exemplary embodiments and with reference to the accompanying diagrammatic drawings, in which:

FIG. 1 shows a developed mesh structure of an exemplary embodiment of the medical device in accordance with the invention, in particular a stent, with 3 cells per circumferential segment;

FIG. 2 shows an enlarged section of the mesh structure according to FIG. 1 in the region of a central cell with a connecting strut, wherein the strut widths S1-S4 are drawn in;

FIG. 3 shows a developed mesh structure of an exemplary embodiment of the medical device in accordance with the invention, in particular a stent, with 2 cells per circumferential segment;

FIG. 4 shows a diagrammatic comparison of a mesh structure with a large number of cells per circumferential segment (left hand side-comparative example) and with a comparatively smaller number of cells per circumferential segment (right hand side—example in accordance with the invention) in a tightly curved state;

FIG. 5 shows a cross section through a mesh structure with 6 cells per circumferential segment and the disposition of the strut connectors on the circumference of the mesh structure (comparative example);

FIG. 6 shows a cross section through a mesh structure with 3 cells per circumferential segment and the disposition of the connectors on the circumference of the mesh structure (example in accordance with the invention);

FIG. 7 shows a developed mesh structure for a further exemplary embodiment of the medical device in accordance with the invention, in particular a stent, with 3 cells per circumferential segment; and

FIG. 8 shows an enlarged section of the mesh structure in accordance with FIG. 7 in the region of a central cell with a connecting strut, wherein the strut widths S1-S4 are drawn in.

### DETAILED DESCRIPTION

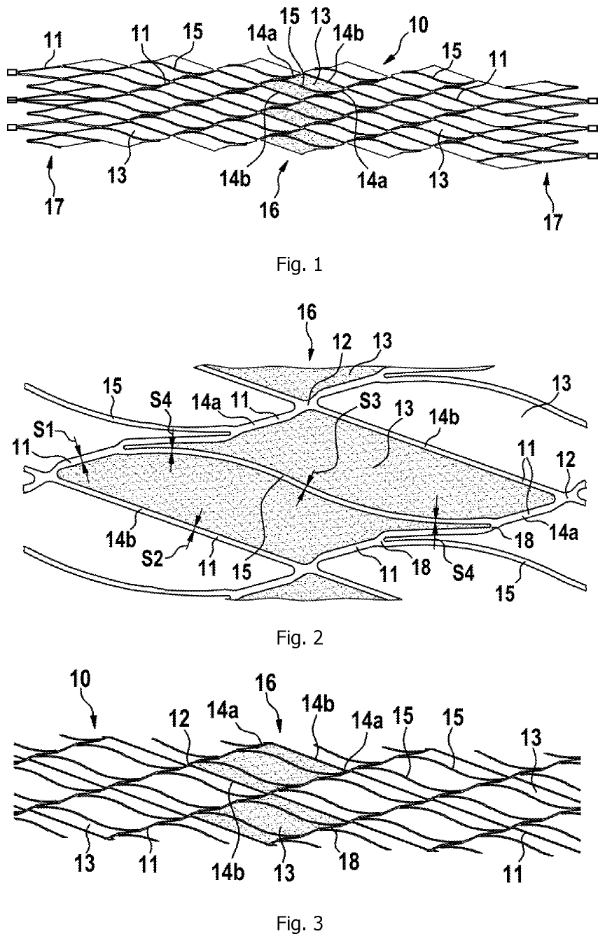

FIGS. 1, 2, 3, 7 and 8 show exemplary embodiments of a medical device in accordance with the invention for introduction into an organ of the body. Here, the application is to use the device as a stent or, in general, as a temporary or permanent vessel implant. Other uses are conceivable, for example as a thrombectomy device. The device has a compressible and expandable mesh structure 10 which is formed from struts 11. As an example, the mesh structure 10 may be produced by laser cutting. For greater clarity, the mesh structure is shown in the developed state in FIGS. 1, 2 and 3.

In use, the mesh structure 10 is tubular.

The struts 11 are connected together by strut connectors 12, which are X-shaped. The struts 11 each delimit a closed cell 13 of the mesh structure 10. In the exemplary embodiment shown here, the cell 13 is rhomboidal in shape. From the figures, it can clearly be seen that this does not mean that a strictly geometrical rhomboidal shape is necessary. The sides of the rhombus could be partially curved and partially straight. The basic shape of the cells 13 is rhomboidal.

The cell 13 is delimited by 4 struts 11 which are each disposed in pairs opposite each other and form two strut pairs 14a, 14b. The opposed struts 11 of the first strut pair 14a are not connected together directly, but with the struts 11 of the second strut pair 14b, they complete the basic rhomboidal shape of the cells 13.

In the example of FIGS. 1, 2, the struts 11 of the first strut pair 14a are curved. The struts 11 of the second strut pair 14b are straight. Other basic shapes for the cell 13 are possible, as long as the cell 13 is closed, as shown in FIGS. 1, 2.

The strut width of the struts 11 of the first strut pair 14a is greater than the strut width of the struts 11 of the second strut pair 14b. This means that the struts 11 of the first strut pair 14a have a higher bending strength than the struts 11 of the second strut pair 14b. The rotation of the cell 13 caused by the expansion and compression of the mesh structure 10 leads to an excellent bending flexibility for the entire mesh structure 10 which distinguishes the mesh structure from other closed cell designs significantly. The mechanism is described in detail and protected in the Applicant's patents EP 2 667 831, DE 10 2013 104 550 B4 and DE 10 2013 107 258 B4 and will not be gone into in further detail here.

In general, the features and functions described in conjunction with a cell 13 of the mesh structure 10 are also disclosed and claimed for other cells 13 of the mesh structure 10. In this manner, circumferential segments 16 and axial sections of the mesh structure consisting of a plurality of circumferential segments 16 may have corresponding cells 13. The edge cells of the mesh structure usually have another design because there, they have to have other functions, such as flaring.

The mesh structure 10 overall has a closed cell design. All of the cells 13 of the mesh structure 10 are closed. Other embodiments of the mesh structure 10 are possible.

As can clearly be seen in FIGS. 1, 2, one of the two strut pairs 14a, 14b, specifically the first strut pair 14a, is connected together by a connecting strut 15. The connecting strut 15 extends into the cell 13 and bridges the cell 13. In other words, the connecting strut 15 is entirely in the plane of the cell 13, which is delimited by the two strut pairs 14a, 14b. For greater clarity, the plane of the cell 13 under consideration is shown with a gray background.

The connecting strut 15 divides the cell 13 into two sub-cells or part-cells, in particular into two half-cells, specifically two substantially symmetrical half-cells, which are disposed between the struts 14a, 14b and the connecting strut 15. For its part, each part-cell also forms a closed cell. The divided cell 13 in this regard forms the main cell which determines the design of the mesh structure 13. The main cell is rhomboidal and has the X-shaped strut connectors 12 at the points of the rhomboid. Specifically, four X-shaped strut connectors 12 are provided per main cell.

FIG. 1 clearly shows that the cells 13 of a first circumferential segment 16 (gray) are all constructed in the same manner and each has a connecting strut 15. FIG. 1 also shows that the adjoining circumferential segments 17 in the axial direction are configured in accordance with the first circumferential segment 16, so that there is a region of cells 13 extending in the axial direction which has the same constructional features and functions or properties. The edge cells are also rhomboidal, but are constructed without connecting struts 15. Other configurations for the mesh structure 10 are possible.

The connecting strut 15 is curved, in particular S-shaped. Other shapes for the connecting strut 15 are possible.

FIG. 2 clearly shows that the connecting strut 15 bridges the cell 13 substantially diagonally. The diagonal disposition of the connecting strut 15 means that the position of the connecting strut 15 deviates from at least one of the two axes, in particular from both axes of the cell 13; specifically, intersects or crosses them. The axes of the cell 13 are not shown in FIG. 2 and comprise the longitudinal axis and the normal axis of the cell 13. The longitudinal axis extends between the two strut connectors 12 of the cell 13 which are farthest apart. The normal axis is shorter than the longitudinal axis. This is the case for every rhomboidal cell 13. The longitudinal axis of the cell 13 runs substantially parallel to the stent axis. In the dilated state of the cell, the shape of the cell can, however, change in a manner such that the cell 13 is higher than it is wide, and also it turns somewhat depending on the state of dilation, so that it is not possible to state that it is strictly “parallel to the stent axis”.

The term “diagonal” is not intended to indicate a strictly geometrical diagonality.

By means of the diagonal disposition of the connecting strut 15, in sections, specifically in the central region of the connecting strut 15, the connecting strut 15 runs approximately parallel to the struts 11 of the second strut pair 14b. This is the case for the expanded state at least. The S-shaped connecting strut 15 is disposed in a manner such that it approaches the respective strut 11 of the first strut pair 14a substantially tangentially.

In the exemplary embodiment, the struts 11 of the second strut pair 14b are straight. The curved connecting strut 15 extends substantially in the same direction as the straight struts 11 of the second strut pair 14b.

The relative disposition of the connecting strut 15 in the cell 13 may change as a function of the state of compression or expansion of the mesh structure 10.

FIGS. 1, 2 show the developed mesh structure 10, i.e. the cutting pattern, showing how the structure is cut. The tubular mesh structure 10 is dilated so that the cells 13 become shorter and higher. The cell angle, i.e. the angle between the strut pairs 14a and 14b, increases here. In the fully expanded state, i.e. when the mesh structure 10 is released, the cell angle is between approximately 100° and 110°; in the vessel, it is between 20° to 30° at the lower permissible operational diameter and 90° at the upper operational diameter. Other cell angles are possible.

The strut width of the connecting strut 15 varies. As can be seen in FIG. 2, the strut width in the region of the axial ends of the connecting strut 15 is smaller than the strut width of the connecting strut 15 in the central region, i.e. in the region which extends between the two ends of the connecting strut 15.

The strut width in the region of the ends is indicated by S4 and in the central region by S3. By this means, on the one hand, the supporting force of the connecting strut 15 in the region of the strut width S3 is increased and on the other hand, the bending flexibility in the region of the attachment (strut width S4) is improved.

The connecting strut 15 is attached by means of a Z-shaped strut connector 18. The Z-shape of the strut connector 18 means that the end of one connecting strut 15 and the end of the next connecting strut 15 in the adjoining cell together with the strut 11 with which the two strut connectors 15 are connected form a kind of Z. The curvature, in particular the S-shaped curvature of the connecting strut 15, means that there is an almost tangential approach of the end of the connecting strut 15 to the strut 11 in the region of the Z-shaped strut connector 18.

In other words, the two horizontal limbs of the Z are curved and approach the strut 11 approximately tangentially. In doing so, the strut width tapers from S3 in the central region to S4 in the end region of the connecting strut. Then it increases again going towards the connector.

Both the crimping capability as well as the wall apposition can be optimized by means of the position, length and strength of the reduction in the strut width (taper).

The strut width S1 of the strut 11 is larger than the strut width S4 of the connecting strut 15 in the region of the strut connector 18. This is advantageous but not obligatory.

The connecting strut of the strut connector 18 between the two adjoining connecting struts 15 has substantially the same strut width S1 as the remaining strut 11 of the first strut pair 14a to which the connecting strut 15 joins.

The Z-shaped strut connector 18 means that the connecting struts 15 of the two adjacent cells 13 do not align with each other. Rather, their connecting positions with the cell 13 are offset with respect to each other in a manner such that in the compressed state, sections of the immediately adjacent connecting struts 15 overlap. In this manner, the crimping capability of the device or the stent is improved, so that in the compressed state, small diameters for the mesh structure 10 can be obtained.

In addition, the Z-shape of the strut connector 18 contributes to the bending flexibility of the mesh structure 10. Z-shaped strut connectors 18 have a greater flexibility than X-shaped strut connectors 12. The flexible attachment of the connecting strut 15 via the strut connector 18 is particularly advantageous. The advantage is even greater because of the tapered ends of the connecting struts 15.

The overlapping of the connecting struts 15 in the compressed state may be adjusted by the offset between the junction positions or connecting positions of the connecting struts 15 and the strut 11 of the first strut pair 14a. The offset between the junction position or connecting position of the connecting struts 15 (i.e. the length of the overlap) may be between 0.1 and 0.9 times the strut length of the strut 11 of the first strut pair 14a of the cell 13, in particular between 0.2 and 0.8 times, in particular between 0.3 and 0.7 times.

The constructional features and properties described in conjunction with the connecting strut 15 are also disclosed and claimed in conjunction with the correspondingly constructed connecting struts 15 of the other cells 13.

The reinforcement of the cell by the connecting strut 15 enables the mesh structure 10 to be modified to the extent that the bending flexibility can be enhanced without the radial force or supporting force of the mesh structure 10 being compromised in the implanted state. Rather, by means of an appropriate design of the connecting struts 15, the supporting force can be evenly distributed onto the vessel wall along with an increased bending flexibility.

The increase in the bending flexibility may, for example, be achieved by reducing the number of cells 13 per circumferential segment 16. This leads to a configuration of the mesh structure 10 which, as shown in FIG. 1, has at least one section, in particular in the central region of the device with a reduced number of cells 13, in particular 3 cells per circumferential segment 16. A further section, in particular the edge regions of the device, has more cells 13, in particular 6 cells per circumferential segment. It can generally be stated that with the invention, the number of cells 13 per circumferential segment 16 can be reduced.

The function of the diagonal struts consists in the fact that the lost supportive effect due to this reduction in the number of cells is compensated for in the interstices without compromising the bending flexibility, however.

In the initial example of FIG. 1, 3 cells per circumferential segment are provided in the central region of the device. Another number of cells is possible, but the bending flexibility will reduce as more cells are provided per circumferential segment.

FIG. 3 shows a further exemplary embodiment in accordance with the invention, wherein 2 cells 13 are provided per circumferential segment 16. It should be noted that the cells 13 of a circumferential segment 16 each have two connecting struts 15 which each bridge the cells 13 substantially diagonally. The attachment of the connecting struts 15 is produced by Z-shaped strut connectors 18. The connecting struts 15 divide the cells 13 into three sub-cells or part-cells which are disposed between the struts 14a, 14b and the connecting struts 15. For its part, each part-cell also forms a closed cell. The divided cell 13 therefore forms the main cell which determines the design of the mesh structure 10. In this specific exemplary embodiment, the strut connectors 12 of the first circumferential segment 16 in the expanded state of the mesh structure are offset on the circumference by an angle of 180°. The shape of the main cell in FIG. 3 corresponds to the shape of the main cell in FIG. 2. Reference should therefore be made to the description provided above.

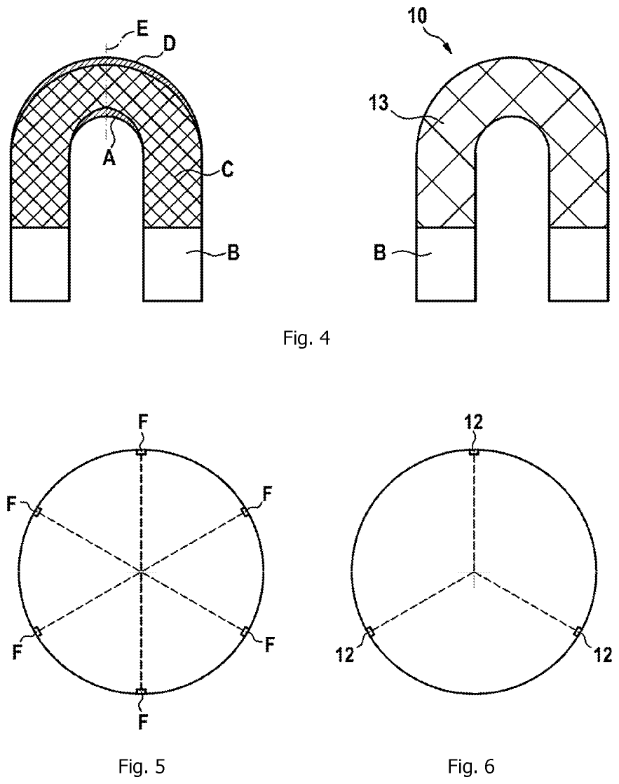

The effect of the exemplary embodiment in accordance with the invention of FIGS. 1 and 2 is shown in FIG. 4.

The left hand view of FIG. 4 diagrammatically shows a narrow-mesh stent C with a 6-cell structure, i.e. six cells per circumferential segment, as a comparative example. The stent C is disposed in a standardized U-bend B as a model of a vessel. Below a specific radius of curvature of the inner curve A, the stent structure lifts off the outer curve D of the bend B because the stent cells cannot stretch any further. The stent C kinks at the inner curve A because the cells can no longer be compressed.

The wall apposition, i.e. the contact of the closed cell stent C with the vessel wall, therefore reduces both on the inside and also on the outside of the vessel with increasing curvature. In this regard, the curvature is the reciprocal of the radius of the central line. On the outside, the rhomboidal cells are stretched more and more in the axial direction until they cannot stretch any further and lift from the vessel wall. In contrast, on the inside, initially the rhomboids are compressed, and then finally, the structure collapses into the interior of the vessel lumen.

In the right hand view of FIG. 4, a stent in accordance with an exemplary embodiment of the invention with a 3-cell structure is shown diagrammatically. The effects shown in the left hand view do not occur, or occur to a lesser extent, in the U-bend B with the same radius of curvature. This is due to the improved bending flexibility of the mesh structure 10 because of the comparatively smaller number of cells 13 per circumferential segment. The smaller number of cells 13 per circumferential segment is possible because of the connecting struts 15 (not shown in FIG. 4), which are disposed in the cells 13 under consideration and increase the radial force of the mesh structure 10. The low supportive effect associated with the small number of cells 13 per circumferential segment is compensated for by the connecting struts 15 and may even be increased.

FIG. 5 shows the cross section E from FIG. 4, left hand view, i.e. the illustration of the struts of a 6-cell structure as a comparative example. This is shown opposite FIG. 6, which shows a corresponding cross section through the stent C from FIG. 4, right hand view, i.e. the illustration of the struts of a 3-cell structure as an exemplary embodiment in accordance with the invention.

When flexed about an axis lying transversely to the longitudinal axis of the stent, the 3-cell design has a better bending flexibility than a 6-cell design of corresponding dimensions. This can best be seen by considering the strut cross sections in a plane which is orthogonal to the longitudinal axis, which lies in a test structure (U-bend) at the point of maximum curvature; see FIGS. 5, 6.

In the 6-cell design, each connector F has exactly one further connector F diametrically opposite it, see FIG. 5. This means that during bending about an axis transverse to the longitudinal axis of the stent, a connector F which lies on the outer line of the bend and is loaded with the maximum tension, lies exactly opposite to a connector F on the inner line of the bend which is loaded with maximum compression. As a result, a high bending resistance moment is generated against the bending.

In contrast, with a 3-cell design, for the same orientation of the strut connector 12 lying on the outer line of the bend, there is no directly opposite strut connector, but the two other strut connectors 12 are respectively offset by 120°, see FIG. 6. A pure 3-cell design, however, would have cell openings which were too large to be able to fulfil the function of a stent for stenosis or for stent-assisted coiling. In contrast, the connecting struts 15 provide for a good supportive effect without increasing the bending stiffness, as is the case with a 6-cell design.

The attachment of the connecting struts 15 to the continuous struts 11 of the 3-cell structure is advantageously carried out via Z-shaped strut connectors 18, which permit a further expansion of the mesh structure 10, in particular on the outer line of the bend, than if the neutral lines of adjacent diagonal connecting struts 15 were to be directly aligned. The strut width of the connecting struts 15 may reduce locally in the vicinity of the Z-shaped strut connectors 18 in order to obtain an optimal deformation of the connecting struts 15 for the application. In particular, by this means, the wall apposition of the connecting struts 15 on the inner curve of the bend can be further improved.

Although the cell structure of the exemplary embodiment in accordance with the invention does have wider struts 11, it requires fewer than with a corresponding 6-cell structure with the same radial force, and so comparable stents can be delivered through the same or even through a smaller catheter lumen.

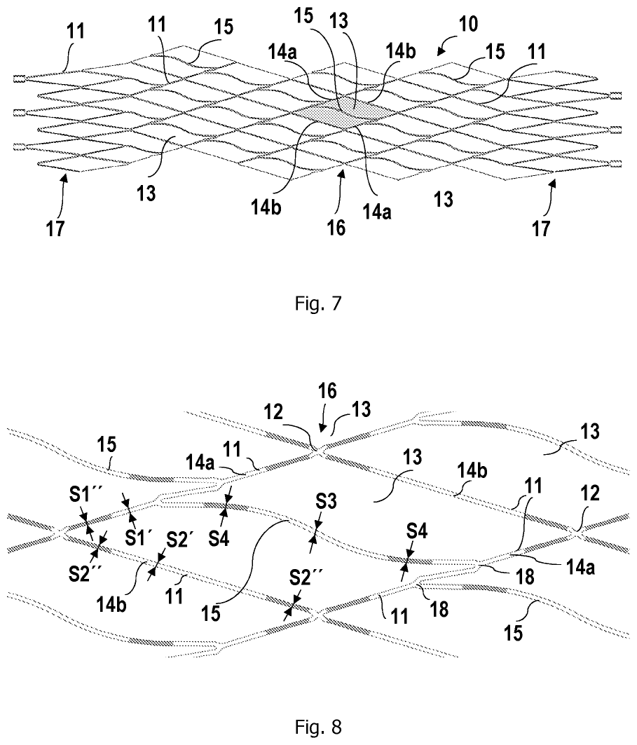

FIG. 7 shows a further exemplary embodiment in accordance with the invention, wherein 3 cells 13 are provided per circumferential segment 16. The cells 13 of a circumferential segment 16 each have a connecting strut 15 which respectively bridges the cells 13 substantially diagonally. It should also be noted that the cells 13 of the adjoining circumferential segments (edge segments) 17 in the axial direction are also rhomboidal, but are constructed without connecting struts 15. It is conceivable for the cells 13 of the edge segments 17 of the proximal end of the mesh structure 10 and the cells 13 of the edge segments 17 of the distal end to be configured differently (not shown). In this manner, the cells 13 of the proximal and distal ends of the mesh structure 10 may have different properties. Thus, for example, flaring at the proximal end of the mesh structure 10 may be stronger than at the distal end of the mesh structure 10.

The strut width of the struts 11 of the first and second strut pair 14a, 14b varies. As can be seen in FIG. 8, the strut width of the struts 11 in a region close do the X-shaped strut connector is smaller than the strut width of the struts 11 in a region close to the Z-shaped strut connectors. The strut width of the struts 11 of the first strut pair 14a in the region close to the X-shaped strut connector is indicated by S1″ and in the region close to the Z-shaped strut connector by S1′. The strut width of the struts of the second strut pair 14b in the region close to the X-shaped strut connector is indicated by S2″ and in the region close to the Z-shaped strut connector by S2′. In this manner, on the one hand, the supporting force of the struts 11 is increased in the region of the strut widths S1′, S2′, and on the other hand, the bending flexibility in the region of the attachment or of the X-shaped strut connector (strut width S1″, S2″) is improved.

The ratio of the strut width S1′ to the strut width S1″ as well as the ratio of the strut width S2′ to the strut width S2″ is between 0.5 and 1.0, such as approximately 0.75, for example. The ratio of the strut width S3 to the strut width S4 of the connecting strut 15 is between 0.7 and 1.0, for example 0.95.

The advantages and constructional features described in conjunction with a 3-cell design can in principle also be applied to a mesh structure which has a number other than 3 cells per circumferential segment.

The cell design is in principle suitable for all clinical applications in which a vessel anatomy is to be treated with a permanent implant or a temporarily inserted implant with wall apposition which is as good as possible, without leading to collapse of the structure. These implants, mostly self-expanding, are preferably laser cut from nitinol tubing, brought to their final diameter by heat treatment and electropolished. Other alloys or production processes are conceivable.

In summary, the invention or the exemplary embodiment described here enables a cell to be designed for laser cut stents with closed cells (closed cell design) which reduces the constriction and kinking of the structure in tightly curved vessels which occur in a closed cell design of the prior art beyond a specific curvature. In this regard, the positive properties of the design, capability of crimping to a narrow catheter lumen, resheathability, narrow-mesh covering of the vessel wall are retained to a great extent (covering aneurysm necks), completely retained (resheathability) or even improved (radial force, capability of crimping to a narrow catheter lumen).

### LIST OF REFERENCE NUMERALS

10 mesh structure
11 struts
11a first strut
11b second strut
12 first strut connector
13 cells
14a first strut pair
14b second strut pair
15 connecting strut
16 first circumferential segment
17 second circumferential segment
18 second strut connector (Z-shaped)

What is claimed is:

1. A medical device for insertion into an organ of a body, with a compressible and expandable mesh structure, the medical device comprising:
struts which are connected together by strut connectors and delimit closed cells of the mesh structure, wherein respectively, two of the struts of at least one of the cells are disposed opposite each other and form a first strut pair and a second strut pair, wherein one of the first strut pair and the second strut pair is connected together by at least one connecting strut which extends into the at least one of the cells and bridges it.

2. The medical device as claimed in claim 1, wherein the at least one of the cells is substantially rhomboidal.

3. The medical device as claimed in claim 1, wherein a plurality of the cells is disposed on at least one circumferential segment, in which respectively one of the first strut pair and the second strut pair is connected together by the at least one connecting strut which extends into the at least one of the cells and bridges it.

4. The medical device as claimed in claim 3, wherein the strut connectors of the at least one circumferential segment are offset with respect to each other on a circumference by an angle which is less than or equal to 180°.

5. The medical device as claimed in claim 1, wherein the at least one connecting strut bridges the at least one of the cells diagonally.

6. The medical device as claimed in claim 1, wherein the at least one connecting strut is connected to the first strut pair respectively by a flexible strut connector.

7. The medical device as claimed in claim 1, wherein a width of the at least one connecting strut in a region of its ends is smaller than the width in a region of the at least one connecting strut which extends between the ends.

8. The medical device as claimed in claim 1, wherein the at least one connecting strut of a first one of the cells and the at least one connecting strut of an adjacent second one of the cells are offset with respect to each other in a circumferential direction.

9. The medical device as claimed in claim 3, wherein the at least one circumferential segment has at least two of the cells.

10. The medical device as claimed in claim 3, wherein there are at least two circumferential segments and a number of the cells on a first one of the circumferential segments is smaller than a number of the cells on a second one of the circumferential segments which is disposed proximally and/or distally to the first one of the circumferential segments in an axial direction of the mesh structure.

11. A treatment system with the medical device as claimed in claim 1, and with a catheter in which a guide element is connected to an axial end of the mesh structure.

12. The medical device as claimed in claim 1, wherein the at least one connecting strut is connected to the first strut pair respectively by a Z-shaped strut connector.
